(19) United States
(12) Patent Application Publication
(10) Pub. No.: US 2026/0070862 A1
(43) Pub. Date: Mar. 12, 2026

(54) COMPACT ELECTRICAL HEATER FOR OLEFIN PRODUCTION

(71) Applicant: Lummus Technology LLC

(72) Inventor: Kandasamy M. Sundaram, Houston, TX (US)

(73) Assignee: Lummus Technology LLC, (omitted)

(21) Appl. No.: 19/320,551

(22) Filed: Sep. 5, 2025

Related U.S. Application Data
(60) Provisional application No. 63/691,905, filed on Sep. 6, 2024.

(51) Int. Cl. C07C 4/04; B01J 19/00; H05B 3/44

(52) U.S. Cl. CPC C07C 4/04; B01J 19/0013; H05B 3/44; B01J 2219/00103; B01J 2219/00135; H05B 2203/005

(57) ABSTRACT

An electrical heater system includes a heater block, a plurality of heating element holes transversing the heater block, a plurality of radiant coil holes transversing the heater block, a plurality of electrical heating elements disposed within the plurality of heating element holes, and a plurality of radiant coils disposed within the plurality of radiant coil holes. A process for olefin production includes heating a plurality of radiant coils using an electrical heater, feeding a hydrocarbon mixture into a plurality of inlet tubes, controlling a temperature of a plurality of electrical heating elements to heat a heater block to a desired temperature, cracking the hydrocarbon mixture in the plurality of radiant coils to produce a cracked effluent, collecting the cracked effluent through a plurality of outlet tubes, and cooling the cracked effluent using a heat exchanger.

### BACKGROUND

Ethylene and other olefins are crucial chemical building blocks that form the basis of many industries. Ethylene and other olefins are typically produced through thermal cracking of hydrocarbons. Currently, ethylene is predominantly produced by thermal cracking of hydrocarbons in fired heaters. In prevalent current technologies, heaters supply heat to radiant coils through radiation, whether using fired heaters or electric heaters. Thus, both fired heaters and conventional electrical heaters face the same problem, which is variable heating of the radiant coils by the radiative transfer of heat from the heat source. This results in the part of the radiant coils facing the heater being the hottest. The uneven heating of the radiant coils is undesirable, as even heating will improve efficiency and selectivity of the cracking process. Therefore, a system for heating radiant coils in a more uniform and efficient way will yield an improved thermal cracking process for olefin production.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to an electrical heater system which includes a heater block, a plurality of heating element holes transversing the heater block, a plurality of radiant coil holes transversing the heater block, a plurality of electrical heating elements disposed within the plurality of heating element holes, and a plurality of radiant coils disposed within the plurality of radiant coil holes.

In another aspect, embodiments disclosed herein relate to a process for olefin production which includes heating a plurality of radiant coils using an electrical heater, feeding a hydrocarbon mixture into a plurality of inlet tubes, controlling a temperature of a plurality of electrical heating elements to heat a heater block to a desired temperature, cracking the hydrocarbon mixture in the plurality of radiant coils to produce a cracked effluent, collecting the cracked effluent through a plurality of outlet tubes, and cooling the cracked effluent using a heat exchanger.

Other aspects and advantages of the claimed subject matter will be apparent from the following description and the appended claims.

### BRIEF DESCRIPTION OF DRAWINGS

FIG. 1 is a diagram of an electrical heater system in accordance with one or more embodiments of the present disclosure.

FIG. 2 is a schematic of powdered material used to fill void volume in accordance with one or more embodiments of the present disclosure.

FIG. 3 is a schematic of variable heating zones in accordance with one or more embodiments of the present disclosure.

FIG. 4 is a diagram of an electrical heater system in accordance with one or more embodiments of the present disclosure.

FIG. 5 is a graph of radiation vs constant wall temperature in accordance with one or more embodiments of the present disclosure.

### DETAILED DESCRIPTION

Currently, ethylene is predominantly produced by thermal cracking of hydrocarbons in fired heaters. In a conventional electrical heater approach for olefin production, fired heaters are replaced with electric heating elements. Embodiments disclosed are directed toward producing ethylene using an electrical heater system where radiant coils are placed inside alumina or ceramic blocks and the block is electrically heated. This provides uniform circumferential heat distribution and reduces the maximum metal temperature for the specified coil outlet temperature and severity. This increases the run length of the cracking operation. Alternatively, the capacity can be increased for the same run length. In addition, the plot space is reduced. By suitably choosing the electrical heating system, all types of heating elements including metallic elements and/or ceramic elements can be used.

### Electrical Heater System

Radiation heat transfer is dictated by view factor, also known as shadow factor. In a typical fired heater cracking unit, the view factor depends upon how the radiant coils are arranged in the radiant box relative to burner location(s), which supply the heat. The radiant coils may be arranged in one row or multiple rows. One row yields the lowest shadow factor and multiple rows yields a higher shadow factor for a given set of coils in a radiant box. The shadow factor affects the maximum to average heat flux ratio. A one row arrangement gives the lowest maximum to average heat flux ratio, which is about 1.1-1.3. As a result, in a typical fired heater cracking unit, there is a circumferential variation of tube temperature at a given elevation of a radiant coil. The point facing the burner is hottest and approximately 90° to this location is coldest. The burners are placed symmetrically on both sides facing the radiant coil in the radiant box. Run length is dictated by the hottest value. Hence, fired heaters and conventional electrical heaters have the same problem, which is the portion of the radiant coil facing the electrical heating element or burner being hotter than other portions of the radiant coil. Embodiments disclosed herein utilize a heater block heated by electrical heating elements, where all sides of the radiant coil tubes “see” the heater block, so the view factor is near or equal to 1.0. This is similar to a tube in a tube arrangement. Therefore, average flux and maximum flux are identical and there is no circumferential tube temperature variation in embodiments disclosed, which is not possible in a gas fired heater. This uniform circumferential temperature reduces the radiant coil metal temperature significantly and results in improved heat transfer, as heat transfer depends upon the temperature differential between the metal temperature and gas temperature. For a given design, run length is increased or shorter coil can be used with a view factor of 1.0 compared to lower view factors, allowing for increase in olefin selectivity, higher throughput, or a combination thereof.

In one or more embodiments, the view factor of the radiant coils at a given elevation may be in a range from a lower limit of any of 0.9, 0.91, 0.92, 0.93, 0.94 and 0.95 with an upper limit of 0.96, 0.97, 0.98, 0.99, and 1.00 where any lower limit may be used in combination with any mathematically compatible upper limit. In one or more embodiments, the maximum to average heat flux ratio of the radiant coils at a given elevation may be in a range from a lower limit of any of 1.00, 1.01, 1.02, 1.03, and 1.04 with an upper limit of 1.05, 1.06, 1.07, 1.08, and 1.09, where any lower limit may be used in combination with any mathematically compatible upper limit.

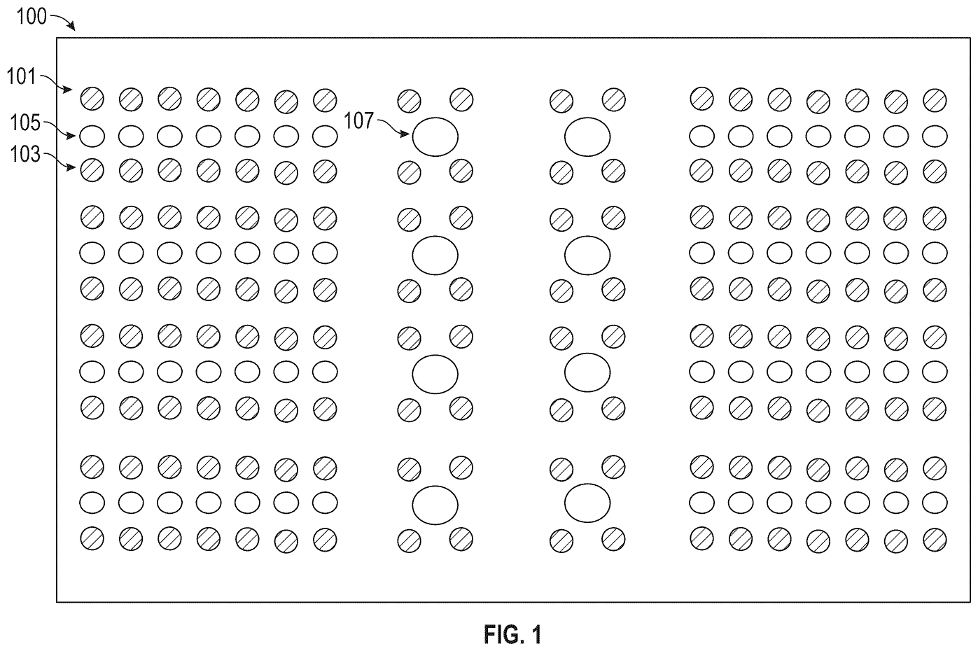

In one aspect, embodiments disclosed herein relate to an electrical heater system for olefin production. The terms radiant coil, coil, and tube are used interchangeably in this disclosure. FIG. 1 shows an electrical heater system 100 in accordance with one or more embodiments of the present disclosure. Heater block 101 contains heating element holes 103, as well as inlet tube holes 105 and outlet tubes holes 107. In one or more embodiments, there are a number of element holes 103, inlet tube holes 105, and outlet tubes holes 107 traversing the heater block. The heater block 101 contains the heating elements within heating element holes 103 and has inlet tubes disposed in inlet tube holes 105 which are connected to outlet tubes disposed in outlet tube holes 107. Though round holes 103, 105, and 107 are shown in FIG. 1, the holes 103, 105, and 107 can also be other shapes, such as square or rectangular or oval. In one or more embodiments, the heating elements disposed in heating element holes 103 heat the heater block 101, which transfers heat to the radiant coils disposed in holes 105 and 107. In such embodiments, the heater block 101 provides uniform heating to the radiant coils embedded within the heater block 101 with no obstruction. Accordingly, no shadow effect would be observed and hence the circumferential temperature is more uniform.

In this disclosure, a shorthand format of X/Y will be used to designate a unit grouping of inlet tubes and outlet tubes, where X number of inlet tubes feed into Y number of outlet tubes. All the outlet tubes are in line with each of the groupings of inlet tubes. In FIG. 1, a 7/1 grouping is shown, in which 7 inlet tubes are connected to 1 outlet tube. This is only for illustration, and other groupings may be used. However, the 7/1 grouping forms the basis of calculation and discussion here. While an arrangement of stacked 14/2 units, with 8 outlet tubes total is depicted in FIG. 1, there is no limit on the arrangements possible. For example, the arrangement could be stacked 14/2 units as shown, or stacked 7/1 units. While 8 outlet tubes are shown, there is no limit to the number of outlet tubes that can be used. For example, 1, 2, 4, 8, 10, 12, 14, 16 or more outlet tubes can be arranged in one, two, or more rows. FIG. 1 shows the tubes arranged in a parallel arrangement with alternating rows of heating elements and radiant coils, with two 7/1 groupings arranged in one row. This grouping is repeated 4 times in FIG. 1. Although illustrated as four parallel groups in FIG. 1, other arrangements are possible.

Depending upon the capacity desired, as many rows as required can be added. In the example shown in FIG. 1, eight 7/1 coils are used. As another example, using a 10/1 grouping may yield double the capacity. 1/1 to 14/1 are also envisioned, and any combination can be used with the process disclosed. Instead of one group of coils with eight 7/1 groupings shown in FIG. 1, sixteen groupings of 7/1 can form the heater. In that case, a single row will have 4 modules of 7/1 type and each heater block will be adjacent to each other. In this way, the capacity of the electrical heater system can be varied as desired.

Where the size of a single alumina block is finite and limited, many blocks will be used. The heater block can be segmented, and many segments can be joined. Since heating elements are placed symmetrically, each block or segment will be heated uniformly. Since the typical length of a radiant coil is over 10 meters, multiple heater blocks will be stacked to achieve this length. For example, if each block is 1 meter in height, ten heater blocks may be stacked to achieve the 10 meter length. Also, shorter blocks could be used, for example, 20 blocks of a 0.5 meter height could be stacked to achieve the 10 meter length.

In one or more embodiments, the heater block includes a material selected from the group consisting of ceramics, metals, and combinations thereof. Ceramics, such as alumina, or other high temperature material such as a high temperature firebrick may be used as the heater block material. Metals and combinations of ceramic materials with metals may be considered in some cases.

FIG. 1 shows a heater block arrangement using two-pass radiant coils, however there is no restriction on the type of radiant coil that can be used in this invention. Similar to a fired heater, there is no radiant section and convection section, only a radiant section. As the same type of coils are used for carrying out the reaction, all coils are referred to as radiant coils. SRT-1 (serpentine) coils to SRT-7 coils (high selective two pass coils), U coils, W coils, M coils and/or single pass coils can all be used. In FIG. 1, each inlet tube is disposed between two heating elements and each outlet tube is disposed between 4 heating elements. However, the number of heating elements for each radiant coil section can be adjusted as required. For example, there may only be 1 heating element per inlet tube, positioned in rows so that there is only 1 row of heating elements per inlet tube. Alternatively, there may be 3 or more heating elements per inlet tube. Also, for example, more or less than 4 heating elements may be used per outlet tube.

Since the heating elements are spaced close together, the heater block is compact. In one or more embodiments, the heating elements are positioned in a range from a lower limit of any of 0.25, 0.5, 1, 1.25, 1.5, and 2 inches apart to an upper limit of any of 24, 25, 26, 27, 28, 29 and 30 inches apart, where any lower limit can be used in combination with any mathematically compatible upper limit. In one or more embodiments, the diameter of the heating elements holes is in a range from a lower limit of any of 0.25, 0.5, 0.75, and 1 inches, to an upper limit of any of 4, 5, 6, 7, 8, and 9 inches, where any lower limit can be used in combination with any mathematically compatible upper limit. In one or more embodiments, the diameter of the inlet tube holes is in a range from a lower limit of any of 0.5, 1, 1.5, and 2 inches, to an upper limit of any of 8, 9, 10, 11, 12, and 13 inches, where any lower limit can be used in combination with any mathematically compatible upper limit. In one or more embodiments, the diameter of the outlet tube holes is in a range from a lower limit of any of 0.5, 1, 1.5, and 2 inches, to an upper limit of any of 8, 9, 10, 11, 12, and 13 inches, where any lower limit can be used in combination with any mathematically compatible upper limit. In one or more embodiments, the inlet tube holes in one row are positioned in a range from a lower limit of any of 0.5, 1, 1.5, and 2 inches apart, to an upper limit of any of 12, 13, 14, 15, 16, 17, and 18 inches apart, where any lower limit can be used in combination with any mathematically compatible upper limit. The compact nature of the heater block reduces the space requirement for heater system installation. Not shown, in one or more embodiments, surrounding the alumina block is the traditional firebox with insulation for protecting the outside environment.

Heating is carried out symmetrically so that the block is heated uniformly and nearly the same temperature is maintained everywhere. Depending upon the heat flux limitation of the electrical elements, the number of holes that use electrical elements can be adjusted. Typically for metallic elements, 35 Kw/m² is used as the heat flux limitation, based on the ceramic surface area that is holding the element. Silicon carbide elements can have a heat flux limitation as high as 150 Kw/m². Each radiant coil can be heated to a specific temperature by independently controlling the temperature of the electrical heating elements near each radiant coil, or many coils can be grouped in one heater block.

The heater blocks may be split into many segments and heating of each section can be controlled individually by adjusting electrical heaters in each section. In this case, each heater block segment temperature will vary. This will result in a different temperature for coils in each section. Through controlling the temperature individually of each heater block segment, an optimum temperature profile for the desired cracking process can be implemented (different feeds fed to different coil sections may have different optimum cracking temperatures, for example). However, this segmentation and variable heating is not required for operation and the entire heater block can be heated uniformly.

FIG. 1 represents a single pass 56/8 arrangement, which produces approximately 45 kilotons/annum (KTA) ethylene. Using a 8/1 grouping will give 50 KTA of ethylene in a similar arrangements for a heater block with 8 output tubes for a total of 64/8 in a heater block. Other groupings such as 12/1 and as large as 14/1 may be used. With this approach a single set of coils within one heater block may have more than 50 KTA and possibly as much as 100 KTA from a single heater block. The flow is distributed to inlet tubes by critical flow venturis and hence there is no issue with flow distribution. Since the block and temperature control can be divided into two or more zones, the outlet tubes can be sequestered in a separate heater block, or separate zone with the same heater block. In this way, inlet and outlet tubes can be heated to different temperatures, with or without thermal insulation. Metallic elements may be used since the flux requirement is reduced significantly. The flux requirement will also be reduced if the heating element diameter is increased, or three heating elements are used per inlet tube as opposed to using two heating elements. The heat flux requirement for the outlet tubes can also be reduced by using larger or more numerous heating elements. The tube spacing can be increased or decreased as required to accommodate larger or more numerous heating elements. In this example, a two-pass coil is described. However, single pass to multi-pass coils with as many as 20 passes can be used. With muti-pass coils, the residence time is increased and thus the olefin selectivity is decreased.

Where there is a void volume between the heater block and radiant coils, as shown in FIG. 1, heat transfer to the radiant coils occurs only by radiation from the heater block. In one or more embodiments, some convective heat transfer may also be added. A flow of air may be passed through the void volume in the radiant coil holes, adding convective heat transfer. The temperature of the air may be adjusted or managed to achieve a desired heat transfer. In this type of embodiment, energy is transferred to the air which may be recovered using convective coils above the alumina block disposed near the openings of the radiant coil holes. The flow of air through the block may be carefully controlled. By sealing the inlets of heating element holes, air will selectively pass through radiant coil holes. This will increase the convective heat transfer component in the radiant coil holes. The advantage of added convective heat transfer is to reduce the maximum metal temperature of the radiant coils since the overall heat transfer coefficient is increased. Conversely, radiant coil holes may be partially blocked so that the air preferentially passes through heating element holes. This will cool the heating elements. When air flows through a heating element hole, that energy may be recovered using convective coils as mentioned above. Use of convective heat transfer is only optional. With a convection section added to the top of the radiant section, the temperature of the process fluid entering the radiant section, known as the cross over temperature, may be adjusted to achieve a desired value. The convection section will contain preheating/superheating coils for process fluids using the hot air passing through heater blocks. Since air is also heated, the total electric power will increase. This additional power is then used for preheating the fluid, preventing waste of the added power. If no air is used, the process fluid may be heated to a desired cross over temperature by other methods such as preheating, either in a secondary transfer line exchanger or by a separate electric heater. Overall energy consumption for the process will not increase, as only the electric power to the radiant section will increase. Other heat duties, such as that of the secondary transfer line exchanger or the electrical preheater, may decrease. FIG. 1 shows only the radiant section.

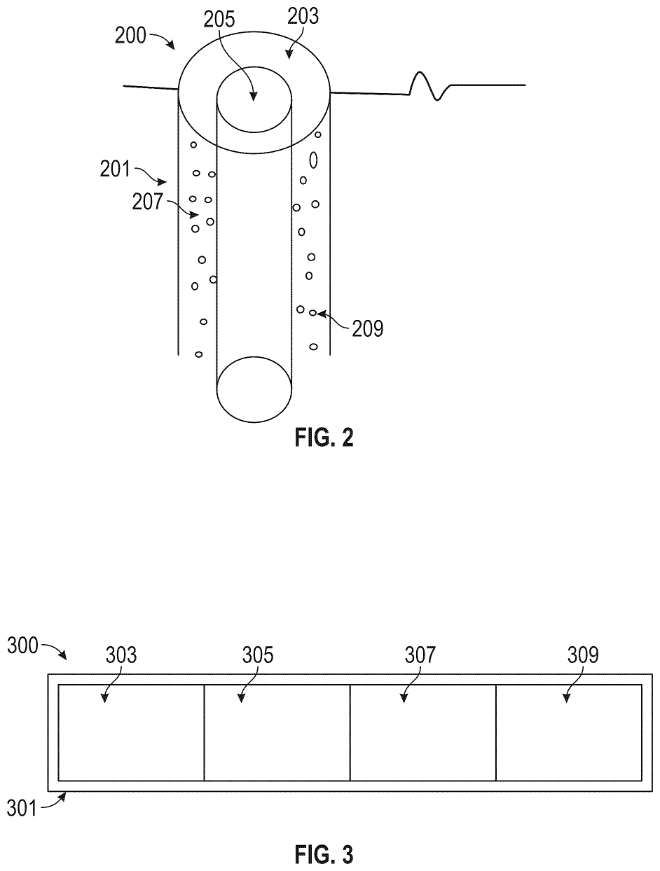

As shown in FIG. 1, the heater block and the radiant coils typically have a gap, such as about 1 to 2 inches, which produces a void volume in the radiant coil holes once the radiant coil is inserted. In one or more embodiments, this void volume can be filled a with highly thermally conductive powdered material, like graphite, quartz, silica, pumice, alumina, and combinations thereof. Alternatively, the tube can be inserted in the hole with almost no void volume, resulting in a tight fit where the walls of the heater block touch the walls of the radiant coil. However, this configuration can introduce strain in the block as the radiant coils contract and expand due to thermal fluctuation, which can damage the block. In one or more embodiments, filling the void volume with a thermally conductive powdered material or inserting the tube with almost no void volume will result in the addition of conductive heat transfer. With these forms of conductive heat transfer, the tube either directly contacts the heater block resulting in direct conduction or the powdered material is heated by radiative and conductive heat transfer from the heater block which then conductively heats the radiant coils. FIG. 2 illustrates a powdered material, such as graphite, being used to fill the void volume in a radiant coil hole. This example 200 shows heater block 201 with radiant coil hole 203, where radiant coil 205 is inserted into radiant coil hole 203. The space between the radiant coil 205 and the walls of the heater block 201 in the radiant coil hole 203 result in a void volume 207. This void volume is filled with a powdered material 209, such as graphite, quartz, silica, pumice, alumina, and combinations thereof.

In a radiative heating mode, where the heat is transferred radiatively from the heater block to the radiant coils, radiant coil wall temperature varies from inlet to outlet. In the conductive heating mode, where a powered material is used to fill void volume in the radiant coil holes, in addition to radiative heat transfer, conduction occurs, and a higher wall temperature and thus higher process gas temperatures are possible.

While FIG. 1 shows only one group of coils fabricated in a block, there is no limitation in the number of blocks that may be connected. FIG. 3 shows four heater blocks connected together as a single larger heater block 300. These connected blocks may be housed in a single casing 301, as shown in FIG. 3, or may be isolated and/or insulated from one another. The different coil groups housed in each block, coil group 1 303, coil group 2 305, coil group 3 307, and coil group 4 309, may be individually controlled to separate temperatures, or may share a temperature control.

In this heater block 300, since each group may have, for example, an output of approximately 45 KTA of ethylene, four groups will produce about 180 KTA of ethylene. Even more than four heater blocks may be combined to increase KTA ethylene and hence with 4-6 groups which can be adjusted larger in size as necessary, as much as 300 KTA or more can be built into a single combined unit. Each group may still be independently controlled.

Split cracking, or cracking multiple hydrocarbons in a single process, is challenging with traditional fired heaters. In a traditional fired heater, the firebox temperature is nearly constant. With embodiments disclosed herein, there is no restriction on the variability of heating. Each individual group of coils can be controlled separately. For example, one coil can crack naphtha, another can crack ethane, a third can crack gasoil, and a fourth can be decoking. Individual control also permits steam-air decoking of individual coils. Housing the groups together or separately will only matter for maintenance and repair or cost implications. For routine operations, one group or heater as spare is sufficient. When the reliability is high, a single coil group as spare is adequate even if housed with other coil groups.

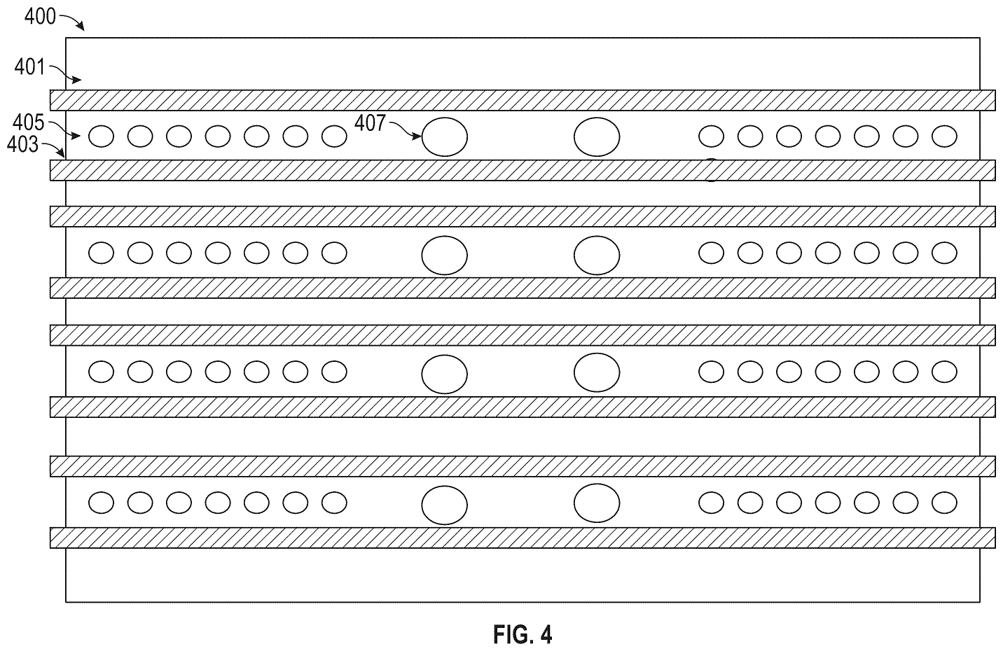

Any type of electrical heating elements may be used. Metallic, silicon carbide, molybdenum alloy-based elements, and high flux heating elements may be used. However, long silicon carbide rods cannot be easily fabricated. The maximum available length for a silicon carbide element is around 5 m. Therefore, instead of using the heating elements vertically as shown in FIG. 1, the heating elements may be inserted into the heater block horizontally, allowing the 5 m length restriction for silicon carbide elements to be met. FIG. 4 shows a heater block system 400, where heating elements holes 403 pass through heater block 401 perpendicular to inlet tube holes 405 and outlet tubes holes 407. While this arrangement allows for the use of short, silicon carbide heating elements, metallic heating elements can also be used. In addition to metal rods, spiral wound metal coils can also be used, both vertically and horizontally.

In addition to the orientation shown in FIG. 4, the heating elements may also be rotated 90 degrees so that they pass between the radiant coils. In this case, the tube spacing for radiant coils may have to be increased to allow room for the heating elements. Though these arrangements may be used for silicon carbide elements due to their length limitation, these arrangements can also be used with metallic heating elements, and heating elements can be interchanged.

During operation, there is a possibility a radiant coil may rupture, and the process gas will leak into the heater block. However, empty space is low and hence oxygen is limited, leading to limited hydrocarbon combustion. Accordingly, the escaped gas will mostly thermally crack and leave the heater block. After leaving the block, there is no additional heat source and the temperature will be reduced. Since multiple heater blocks are combined to a single unit, a pilot light can be installed to provide a flame source without the outer housing for combustion as an additional feature for a whole unit of combined blocks. This reduces the risk of explosion in the unit. Tube rupture and the associated safety handling is not discussed here. That can be achieved similar to any electric heater, including using a stack to vent gases and placing pilot flames near the stack.

In some embodiments, boiler feed water (BFW coils) and/or hydrocarbon plus dilution steam (HC+DS) steam superheat coils may be placed in open space above the block. This can act as additional heating for these streams before entering the radiant section of a heater. The sweep gas under normal conditions will affect the stream (BFW or HC+DS steam) to only a small extent. If a coil ruptures while using BFW coils or HC+DS steam superheat coils, the temperature change will be high as these coils extract maximum heat from air/HC mixture in the open space above the block.

Generally, the materials used for heater blocks are electrically non-conductive, such as alumina. However, the radiant coils which are electrically conductive are isolated from the alumina blocks for higher level of safety when radiation is considered. Since the heating elements are used to heat process fluids and fluids like air to above 700° C. and the elements are in contact with these fluids, safety precautions must be implemented. Accordingly, the whole alumina block can be electrically isolated from the heater casing and radiant coils.

The radiant coils may be inserted in a narrow hole with a small clearance, such as described above for implementing a conductive mode. The radiant coils will bend and bow and may touch the alumina block. In the conductive mode, it is always touching the block. However, in radiant mode, there is clearance. Therefore, to ensure clearance in radiant mode, spacers are used. By using spacers disposed along the radiant coil, the straightness of the radiant coil is maintained for long durations. As an alternative to using spacers, void volume between the radiant coils and the heater block wall in the radiant coil holes may be increased. However, this may increase the alumina block dimensions and heater width, or decrease the heat transfer performance from the alumina block to the radiant coil.

Each radiant coil consumes about 5 to 20 MW of power depending upon the ethylene capacity, however each heater block behaves independently. Therefore, control is based on the performance of an individually controlled section, such as a heater block or group of coils. As a result, severity, feed, and operating conditions may be altered independently for each section or coil. Adjacent heater blocks can be joined or isolated thermally and electrically. When thermally joined the whole group will be operated as one unit. The heater system still produces a small quantity of saturated super high pressure steam. Therefore, a steam drum is required. Generally, one to four conventional or bathtub style exchangers are connected to a single steam drum. Other types of transfer line exchangers may also be considered. The saturated super high pressure steam that is generated may be used to preheat the feed to the radiant coils with little excess saturated super high pressure steam being exported.

The outlet tubes are connected to heat exchangers for cooling effluent gas. In one or more embodiments, a linear exchanger, typically a double pipe exchanger or Schmische Linear exchanger, is used to cool the effluent. Use of wye bends allows multiple outlet tubes to be connected, and conventional shell and tube type heat exchangers can also be utilized. The type of exchanger is not limited, any type of applicable exchanger may be used.

The chosen coil arrangement favors linear (or double pipe) exchangers. Conventional transfer line exchangers can also be considered. Up to twenty linear exchangers may be housed in a single group. 1-6 groups of transfer line exchangers can be connected to a single steam drum. Effluents exiting primary transfer line exchangers can be used to preheat the feed (HC+DS) mixture or dilution steam using secondary transfer line exchangers. Each primary linear exchanger can have a corresponding secondary transfer line exchanger, or a group of primary transfer line exchangers can be combined to have a common secondary transfer line exchanger. The effluents are cooled sufficiently, and both primary and secondary transfer line exchangers can still be steam-air decoked and very rarely require mechanical cleaning. If the feed preheating is not sufficient, a conventional electric heater using convective mode available for low-temperature heating or a compact electric heater discussed here can be used. This way, inlet temperature to radiant section is increased and thereby the compact heater duty will be reduced. Higher duty in the radiant section will increase the tube metal temperature.

In the arrangement shown in FIG. 1, there are no heat flux issues. The process gas temperature increases continuously along the coil length. The heater blocks are maintained at an almost constant temperature. Therefore, the maximum radiant coil process temperature occurs almost at the outlet end. In some cases, the maximum radiant coil process temperature occurs at the first pass end. At both locations, radiant coil metal temperatures can be monitored using skin thermocouples in addition to measuring block temperatures. In fired heaters, peak temperature occurs at the middle of the furnace due to the flue gas temperature profile and hence measuring it with a thermocouple is difficult.

In another aspect, embodiments disclosed herein relate to a process for olefin production, which includes heating a plurality of radiant coils using an electrical heater, where the electrical heater is as described above; feeding a hydrocarbon mixture into a plurality of inlet tubes; controlling the temperature of a plurality of electrical heating elements to heat the heater block to a desired temperature, wherein heat from the heater block is transferred to the plurality of radiant coils; cracking the hydrocarbon mixture in the plurality of radiant coils to produce a cracked effluent; collecting the cracked effluent through the plurality of outlet tubes; and cooling the cracked effluent using a linear exchanger.

Heating the plurality of radiant coils using the electrical heater includes setting the temperature of the electrical heating elements to a desired initial temperature to heat the heater block and transfer heat to the radiant coils. Feeding the hydrocarbon mixture into the plurality of inlet tubes may be achieved through any typical means and can include any type of hydrocarbon mixture applicable to cracking. Controlling the temperature of a plurality of electrical heating elements to heat the heater block to a desired temperature includes adjusting the temperature of the heater block or heater block sections to a temperature that confers the desired temperature to the radiant coils. The heat from the heater block may transfer to the radiant coils through a radiative mode, a conductive mode, or a combination. Convective heat transfer may also be added to the process. The hydrocarbon mixture that is cracked in the plurality of radiant coils produces a cracked effluent, which is collected through the plurality of outlet tubes. The cracked effluent is then cooled using a linear exchanger, however other types of exchangers could be used.

The electrical heater system used for this process is as described above. The process can be adapted for the various embodiments of the heater system shown above. For example, the heater block may be segmented into a plurality of heating zones, the method further including varying a temperature of each of the plurality of heating zones by independently controlling a plurality of electrical heating elements in each of the plurality of heating zones. In one or more embodiments, the temperature of each of the plurality of heating zones is controlled to optimize olefin production. The process may be run in a conductive mode as well, either by filling void volume with a powdered material, or inserting the radiant coils tightly into the heater block. More convective heat transfer may be added to the process by the process further including feeding air through the plurality of radiant coil holes during the controlling the temperature of the plurality of electrical heating elements.

### Example Electrical Heater System

For a 56/8 configuration with a 45 KTA ethylene output, the approximate heater block size is around 66 in×105 in with a height of 45 feet. However, the exact dimensions may vary. In one or more embodiments, the width may be in a range from a lower limit of any of 30, 40, 50, 60, 70, 80 or 90 inches with an upper limit of 100, 110, 120, 130, 140 and 150 inches, where any lower limit may be used in combination with any mathematically compatible upper limit. In one or more embodiments, the length may be in a range from a lower limit of any of 50, 60, 70, 80, 90, 100, or 110 inches with an upper limit of 120, 130, 140, 150, 160, 170 and 180 inches, where any lower limit may be used in combination with any mathematically compatible upper limit. In one or more embodiments, the height may be in a range from a lower limit of any of 1, 2, 3, 4, 5, 6, 7, 8, 9, 10, 20, 30, 40, 50, 60, 70, and 80 feet with an upper limit of 90, 100, 110, 120, 130, 140, 150, 160, and 170 feet, where any lower limit may be used in combination with any mathematically compatible upper limit.

The heater block is insulated, and an outer steel housing is used to protect the outside environment. Example properties and calculations for this example heater block arrangement are shown in Table 1, including the calculated metallic flux which is under 25 kW/m². In this example, the electrical heating element holes need not be spaced evenly or in a pattern. The electrical heating element holes can be arranged in many ways. The block may be segmented. Based on the material, which may be a type of alumina or other firebrick, the number of segments will be decided as well as how they will be bonded. The heater block size can be adjusted. In this example, alumina is used as the block material, but other heater block materials may be used.

TABLE 1
Compact Electric heater

| Feed | Naphtha |
|---|---|
| Coils | 10 |
| Coil arrangement | 56/8 |
| First pass ID (in) | 2 |
| First pass OD (in) | 2.5 |
| Parallel inlet tubes | 56 |
| Second pass ID (in) | 5.5 |
| Second pass OD (in) | 6.1 |
| Length per pass (ft) | 45 |
| Parallel outlet tubes | 8 |
| Hydrocarbon Flow (lb/h/coil) | 42697 |
| Steam to Oil Ratio S/O (w/w) | 0.5 |
| Cross over temperature (° F.) | 1040 |
| Radiant duty (MMBTU/h/coil) | 54.78 |
| Heating element holes/inlet Tube | 2 |
| Heating element holes/outlet Tube | 4 |
| Length of element hole | 45 |
| Total inlet tube heating element holes | 112 |
| Total outlet tube heating element holes | 32 |
| Diameter of inlet tube heating element hole (in) | 4.5 |
| Diameter of outlet tube heating element hole (in) | 4.5 |
| Total heating element hole area (ft²) | 7634.1 |
| Calculated Flux (kW/m²) | 22.63 |

### Example

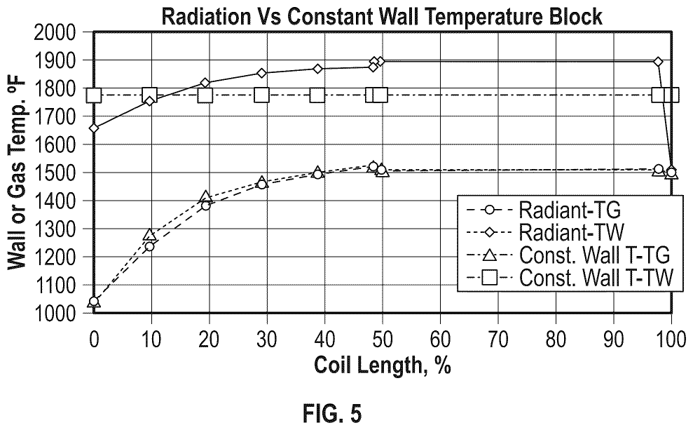

FIG. 5 shows a comparison of gas/radiant coil temperatures (TG) with wall temperatures (TW) versus coil length when operating in either a solely radiative heat transfer mode using a fired heater or when the radiant coil is in close contact with the heater wall of a heater block as described above leading to conductive transfer (Const. Wall T). FIG. 5 compares radiant mode gas/coil temperature (Radiant-TG), radiant mode wall temperature (Radiant-TW), conductive mode gas/coil temperature (Const.Wall T-TG), and conductive mode wall temperature (Const.Wall T-TW). Radiant mode refers to that obtained in a fired heater using fuel gas, whereas conductive mode refers to using a compact electrical heater discussed here. As shown in FIG. 5, the gas/coil temperature is almost the same whether heat is transferred by radiation or by conduction to the radiant coil, comparing radiant gas temperature (Radiant-TG) to constant wall gas temperature (Const. Wall T-TG). However, the maximum heater block wall temperature is significantly reduced for the conductive transfer mode, dropping from 1893° F. to 1775° F., comparing Radiant-TW to Const.Wall T-TW. This will permit the system to operate at high conversion or short residence time by cutting the length of the coil in a conductive mode by increasing the wall temperature. For example, operating at 1825° F. instead of 1775° F. will reduce the coil length per pass from 45 feet to 40 feet and will improve the ethylene yield by 0.15 wt %, and higher yield improvements are possible as long as heat flux is not limiting. The wall temperature and selectivity may be increased further. In an extreme example, single pass coils with ceramic tubes can be used and can operate with a 10-50 millisecond residence time, giving a 5 to 10% improvement in yields. Between the radiant section and the transfer line exchanger there is a small adiabatic section wherein the gas temperature will drop. The duty required for additional feed conversion in this adiabatic section comes from the detectable heat of the reaction mixture and that is the cause of the temperature drop. There is no limitation in coil selection; single pass to multi-pass (8 pass SRT 1) may be used.

Embodiments of the present disclosure may provide at least one of the following advantages. The compact electrical heater in accordance with one or more embodiments of the present disclosure enables reduced plot space and longer run lengths. Ethylene yields may be improved, and any electrical element can be used with the current invention. Metallic elements that have low flux rating compared to silicon carbide elements may be used. In this invention the block is heated by inserting heating elements in many openings, and to meet the flux requirement, the number of openings and heating elements may be increased. The heater blocks transfer the heat to the radiant coils with uniform view factor, unlike conventional fired heating boxes. This invention provides for facile segmentation of the cracking process, where different heater blocks or sections of the same heater block can be heated to different temperatures by independently controlling the temperature of the heating elements. This segmentation can extend down to each individual radiant coil being controlled to its own temperature. With this operation possible, many heater blocks can be grouped together and a protective casing built for all heater blocks in a group, or a protective casing can be built for individual heater blocks.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. An electrical heater system, comprising:
a heater block;
a plurality of heating element holes transversing the heater block;
a plurality of radiant coil holes transversing the heater block;
wherein the plurality of heating element holes and plurality of radiant coil holes are arranged in rows;
a plurality of electrical heating elements disposed within the plurality of heating element holes; and
a plurality of radiant coils disposed within the plurality of radiant coil holes.

2. The electrical heater system of claim 1, further comprising:
a housing surrounding the heater block;
a control system in electrical communication with the plurality of electrical heating elements; and
a heat exchanger positioned adjacent to the heater block, wherein the heat exchanger cools effluent gas.

3. The electrical heater system of claim 1, wherein the plurality of heating element holes and plurality of radiant coil holes are arranged in parallel rows.

4. The electrical heater system of claim 1, wherein the plurality of heating element holes and plurality of radiant coil holes are arranged in perpendicular rows.

5. The electrical heater system of claim 1, wherein a shape of the plurality of heating element holes and a shape of the plurality of radiant coil holes are each independently selected from the group consisting of circular, oval, square, or rectangular.

6. The electrical heater system of claim 1, wherein the plurality of radiant coils comprises 1 to 400 inlet tubes and 1 to 400 outlet tubes, where one or more inlet tubes feed into at least one outlet tube.

7. The electrical heater system of claim 1, wherein the heater block is comprised of a material selected from the group consisting of ceramics, metals, and combinations thereof.

8. The electrical heater system of claim 1, wherein the plurality of electrical heating elements are selected from the group consisting of silicon carbide heating elements, metallic heating elements, molybdenum alloy-based heating elements, high flux heating elements, and combinations thereof.

9. The electrical heater system of claim 1, wherein a view factor of the plurality of radiant coils is in a range of 0.95 to 1.00.

10. The electrical heater system of claim 1, wherein a maximum to average heat flux ratio of the radiant coils at a given elevation is in a range of 1.00 to 1.09.

11. The electrical heater system of claim 1, further comprising a powdered material disposed in a void volume between the heater block and the plurality of radiant coils in the plurality of radiant coil holes, wherein the powdered material is selected from the group consisting of graphite, quartz, silica, pumice, alumina, and combinations thereof.

12. The electrical heater system of claim 1, wherein the heater block is segmented into a plurality of heating zones wherein each heating zone comprises a plurality of heating elements which are controlled independently, allowing for differential heating between heating zones.

13. A process for olefin production, the process comprising;
heating a plurality of radiant coils using an electrical heater, the electrical heater comprising:
a heater block;
a plurality of heating element holes transversing the heater block;
a plurality of radiant coil holes transversing the heater block;
wherein the plurality of heating element holes and plurality of radiant coil holes are arranged in rows in a parallel or perpendicular arrangement;
a plurality of electrical heating elements disposed within the plurality of heating element holes;
a plurality of radiant coils disposed within the plurality of radiant coil holes;
wherein the plurality of radiant coils comprises 1 to 400 inlet tubes and 1 to 50 outlet tubes, where one or more inlet tubes feed into at least one outlet tube;
a housing surrounding the heater block;
a control system in electrical communication with the plurality of electrical heating elements; and
a heat exchanger positioned adjacent to the heater block;
feeding a hydrocarbon mixture into the plurality of inlet tubes;
controlling a temperature of the plurality of electrical heating elements to heat the heater block to a desired temperature;
wherein heat from the heater block is transferred to the plurality of radiant coils;
cracking the hydrocarbon mixture in the plurality of radiant coils to produce a cracked effluent;
collecting the cracked effluent through the plurality of outlet tubes; and
cooling the cracked effluent using the heat exchanger.

14. The process of claim 13, wherein the heater block is comprised of a material is selected from the group consisting of ceramics, metals and combinations thereof.

15. The process of claim 13, wherein a view factor of the plurality of radiant coils is in a range of 0.95 to 1.00.

16. The process of claim 13, wherein a maximum to average heat flux ratio of the radiant coils at a given elevation is in a range of 1.00 to 1.09.

17. The process of claim 13, wherein the heater block is segmented into a plurality of heating zones, further comprising varying a temperature of each of the plurality of heating zones by independently controlling the plurality of electrical heating elements in each of the plurality of heating zones.

18. The process of claim 17, further comprising varying the temperature of each of the plurality of heating zones to optimize olefin production in the plurality of radiant coils.

19. The process of claim 13, further comprising disposing a powdered material into a void volume between the heater block and the plurality of radiant coils in the plurality of radiant coil holes, wherein the powdered material is selected from the group consisting of graphite, quartz, silica, pumice, alumina, and combinations thereof.

20. The process of claim 13, further comprising feeding air through the plurality of radiant coil holes during the controlling the temperature of the plurality of electrical heating elements to heat the heater block.
